(19) United States
(12) Patent Application Publication
(10) Pub. No.: US 2026/0225076 A1
(43) Pub. Date: Aug. 6, 2026

(54) CATALYST AND ASSOCIATED REACTION PROCESS FOR THE PRODUCTION OF WAXES

(71) Applicant: PETROLIAM NASIONAL BERHAD (PETRONAS), Kuala Lumpur (MY)

(72) Inventors: Shamina ABDUL ALEEM, Kajang, Selangor (MY); Zi Kang KOI, Kajang, Selangor (MY); Andrea DOLFI, Villastellone (TO) (IT); Jamali BASAR, Kajang, Selangor (MY); Haslinda MOHD SIDEK, Kota Kinabalu, Sabah (MY); Abdulkareem Ghassan ABDULKAREEM ALSULTAN, Selangor Darul Ehsan (MY); Sivasangar SEENIVASAGAM, Bintulu, Sarawak (MY)

(21) Appl. No.: 19/149,653

(22) PCT Filed: Jan. 22, 2024
(86) PCT No.: PCT/MY2024/050002

§ 371 Date: Jul. 21, 2025

(30) Foreign Application Priority Data
Jan. 30, 2023 (MY) PI2023000493

(51) Int. Cl. B01J 23/00; B01J 21/16; B01J 35/54; B01J 35/61; B01J 35/63; B01J 35/64; B01J 37/00; B01J 37/08; C07C 45/41

(52) U.S. Cl. CPC B01J 23/007; B01J 21/16; B01J 35/54; B01J 35/615; B01J 35/633; B01J 35/647; B01J 37/0009; B01J 37/08; C07C 45/41

(57) ABSTRACT

Disclosed herein is a method of preparing a bio-wax, comprising the steps: (i) providing a feedstock comprising one or more fatty acids, each of the one or more fatty acids having from 12 to 22 carbon atoms; and (ii) subjecting the feedstock to a ketonisation reaction to produce a wax, wherein said ketonisation reaction is catalysed by a catalyst composite material comprising: (a) a layered double oxide; and (b) a binder.

### FIELD OF INVENTION

The present invention relates to a novel catalyst and associated process for converting carboxylic acid and any associated bio-lipids/vegetable or animal oils derived fatty acids into a wax (e.g. a bio-wax or non-bio or partially bio-wax if based upon fossil-derived chemical feedstocks). More specifically, the process involves a solvent-free decarboxylative coupling reaction in which two carboxylic acid molecules react to form a ketone, together with carbon dioxide and water co-products, as opposed to conventional methods where solvent is required. A high-performing catalyst in pellet form encompassing Mg—Al layered double oxide (LDO) as the active component and a binder (e.g. clay) are disclosed. Finally, the catalyst has been rigorously tested under continuous flow packed bed reactor for >500 hours.

### BACKGROUND

The listing or discussion of a prior-published document in this specification should not necessarily be taken as an acknowledgement that the document is part of the state of the art or is common general knowledge.

Paraffin waxes play a pivotal role in a wide range of applications such as candles, packaging, coatings, cosmetics, emulsions, rubbers, phase change materials and lubricants, thus the supply of this substance is in huge demand. However, as the worldwide supply of petroleum diminishes, it becomes urgent to develop an alternative route that can reduce the unsustainable consumption of fossil fuel. In this context, the potential of bio-waxes development and its application has received wider consideration from researchers. Development of sustainable routes to produce bio-based compounds from renewable feedstock is the most relevant strategy to counterbalance the inevitable depletion of fossil resources in the near future.

Production of bio-wax starts with a bio-based feedstock, either from biomass or bio derived lipids. Biomass feedstock primarily includes lignin, starches, lignocellulose, sugars, fats, and oils which is advantageous in producing intermediate chemicals such as esters, acids, ketones and alcohols. Bio-waxes can be produced from many different types of plant oils such as rapeseed, soybean, sunflower, palm and coconut, wax, esters and plant polymeric carbohydrates. For example, palm oil and palm-based bio feed are the main source of feedstock for biofuels (biodiesel, green diesel) and other biochemicals in the oleochemical industry, especially those located in SouthEast Asia. One of the by-products of palm oil extraction is known as Palm Fatty Acids Distillates (PFAD), which accounts for up to 5% of the raw material inputs and considered as an unwanted processing residue and palmitic acid is regarded as the largest fraction of above 45%.

The highly paraffinic structure of PFAD is suitable for conversion to paraffinic hydrocarbon products. However, PFAD's high oxygen containing constituents need to be upgraded into feasible starting material for production of fuel, lubricants or other oleochemical synthesis. One of the catalytic reaction pathway that can be employed in converting these oxygenated compounds is via the ketonization reaction and the use of heterogenous catalysts are vital in this process. Ketonization converts the carboxylic acids to form new C—C bonds via decarboxylative carbon coupling to yield alkanones, carbon dioxide (CO₂) and water (H₂O) (FIG. 1). No hydrogen is required for the ketonization of carboxylic acids.

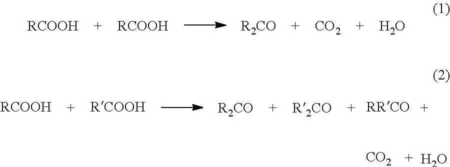

Existing catalysts and ketonization process have a few drawbacks:
i. focus on short chained carboxylic acids. Usually, the feedstock is C₁-C₅ (or less than C₁₂) and not on long-chained carboxylic acids;
ii. process is solvent-intensive-usually requires diluting the lipid/bio-derived fatty acids feeds with solvents like hexane, dodecane, xylene and etc.;
iii the catalysts used are usually monometallic or bimetallic catalysts with moderate to low yields of desired ketones; and
iv. no commercialized catalyst in market that has proven capability to convert long chain carboxylic acid into long chain ketones with superior performance in conversion, yield and selectivity under continuous system in the absence of solvents.

Therefore, there exists a need to formulate a pelletized catalyst that can overcome the drawbacks stated above.

### SUMMARY OF INVENTION

Aspects and embodiments of the invention will now be discussed by reference to the following numbered clauses.

1. A method of preparing a wax, comprising the steps:
(i) providing a feedstock comprising one or more fatty acids, each of the one or more fatty acids having from 2 to 22 carbon atoms; and
(ii) subjecting the feedstock to a ketonisation reaction to produce a wax, wherein said ketonisation reaction is catalysed by a catalyst composite material comprising:
(a) a layered double oxide; and
(b) a binder.

2. The method according to Clause 1, wherein step (ii) is performed in the absence of solvent.

3. The method according to Clause 1 or 2, wherein the one or more fatty acids are selected from monocarboxylic acids and dicarboxylic acids, optionally wherein the monocarboxylic acids have from 12 to 22 carbon atoms; and the dicarboxylic acids have from 2 to 11 carbon atoms.

4. The method according to Clause 3, wherein when the one or more fatty acids are only monocarboxylic acids, then the wax has one or more components having from 23 to 43 carbon atoms, optionally wherein the wax is a bio-wax, further optionally wherein the bio-wax has one or more components having from 23 to 43 carbon atoms.

5. The method according to any one of the preceding clauses, wherein:
the binder is a clay of any type, optionally wherein the clay is a Montmorillonite clay, further optionally wherein the clay is Montmorillonite K30; and/or
the layered double oxide is in the form of a plurality of particles that are distributed homogeneously throughout the catalyst composite material.

6. The method according to any one of the preceding clauses, wherein the catalyst composite material is in the form of a pellet.

7. The method according to any one of the preceding clauses, wherein the layered double oxide is formed from the combination of divalent metal cations, trivalent metal cations and oxygen atoms, wherein:
the divalent metal cations are selected from one or more of the group consisting of magnesium, copper, zinc, nickel, and manganese; and
the trivalent metal cations are selected from one or more of the group consisting of aluminium, iron, and chromium,
   further optionally wherein the layered double oxide is a magnesium-aluminium layered double oxide.

8. The method according to any one of the preceding clauses, wherein the catalyst composite material comprises:
(a) from 30 to 50 wt. % of the layered double oxide; and
(b) from 50 to 70 wt. % of the binder.

9. The method according to any one of the preceding clauses, wherein:
step (ii) is performed in the absence of a solvent; and/or
the binder is a clay of any type, e.g. Montmorillonite clay.

10. The method according to any one of the preceding clauses, wherein step (ii) is performed at a temperature of from 350 to 450° C., such as from 380 to 420° C., e.g. about 400° C.

11. The method according to any one of the preceding clauses, wherein step (ii) is performed at Liquid Hourly Space Velocity (LHSV) of from 0.8 to 1.2.

12. The method according to any one of the preceding clauses, wherein the feedstock is one or more of a fossil fuel-derived feedstock and a biologically-derived feedstock, optionally wherein the feedstock comprises vegetable fatty acid distillate (VFAD) (e.g. palm fatty acid distillate (PFAD)).

13. A catalyst composite material comprising:
(a) a layered double oxide; and
(b) a binder.

14. The catalyst composite material according to Clause 13, wherein the binder is a clay of any type, optionally wherein the clay is a Montmorillonite clay, further optionally wherein the clay is Montmorillonite K30.

15. The catalyst composite material according to Clause 13 or 14, wherein the layered double oxide is in the form of a plurality of particles that are distributed homogeneously throughout the catalyst composite material.

16. The catalyst composite material according to any one of Clauses 13 to 15, wherein the layered double oxide is a magnesium-aluminium layered double oxide.

17. The catalyst composite material according to any one of Clauses 13 to 16, comprising:
(a) from 30 to 50 wt. % of the composite material; and
(b) from 50 to 70 wt. % of the binder.

18. The catalyst composite material according to any one of Clauses 13 to 17, wherein the catalyst composition is in the form of a pellet.

19. A method of forming a catalyst composite material according to any one of Clauses 13 to 18, wherein the method comprises the steps of:
(a) providing a mixture comprising a layered double oxide, a binder, a plasticizer, a lubricant, and a peptizer; and
(b) subjecting the mixture to calcination for a period of time to provide the catalyst composite material.

20. The method according to Clause 19, wherein the plasticizer is a material containing polar and/or non-polar functionality, optionally wherein the plasticizer is water.

21. The method according to Clause 19 or Clause 20, wherein the lubricant is a diol.

22. The method according to any one of Clauses 19 to 21, wherein the peptizer is an acid, optionally wherein the peptizer is an inorganic acid.

### DRAWINGS

FIG. 1 depicts homo ketonization and transketonization reaction.

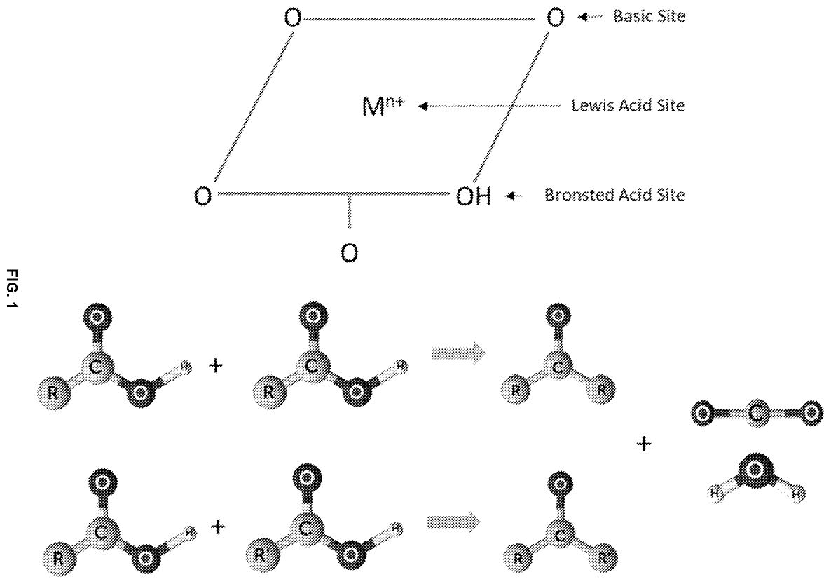

FIG. 2 depicts (a) possible reactions and reaction pathways and (b) field emission scanning electron microscopy (FE-SEM) morphology and characteristics of Mg—Al derived layered double oxide (LDO).

FIG. 3 depicts the field emission scanning electron microscopy (FE-SEM) morphology and energy dispersive X-ray analysis (EDX) of Mg—Al derived layered double oxide (LDO)-clay composite catalyst pellet.

FIG. 4 depicts the N₂ adsorption-desorption isotherm.

FIG. 5 depicts the temperature-programmed desorption (TPD)-CO₂ profile.

FIG. 6 depicts the TPD-NH₃ profile.

FIG. 7 depicts the set-up of a continuous flow packed bed reactor.

FIG. 8 depicts the catalytic performance of Mg—Al derived LDO-clay composite catalyst for >500 hours.

### DESCRIPTION

It has been surprisingly found that some or all of the problems identified hereinbefore may be solved through the use of a catalyst composite material in a method of forming a wax. Thus, in a first aspect of the invention, there is provided a method of preparing a wax, comprising the steps:
(i) providing a feedstock comprising one or more fatty acids, each of the one or more fatty acids having from 2 to 22 carbon atoms; and
(ii) subjecting the feedstock to a ketonisation reaction to produce a wax, wherein said ketonisation reaction is catalysed by a catalyst composite material comprising:
(a) a layered double oxide; and
(b) a binder.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

Any suitable layered double oxide may be used herein. The layered double oxide may be formed from any suitable combination of divalent and trivalent metal cations in combination with oxygen atoms. Suitable divalent metal cations include, but are not limited to magnesium, copper, zinc, nickel, manganese and combinations of said divalent metal cations. Suitable trivalent metal cations include, but are not limited to, aluminium, iron, chromium, and combinations of said trivalent metal cations. In embodiments that may be mentioned herein, the layered double oxide may be a magnesium-aluminium layered double oxide.

Any suitable binder may be used herein, provided that it can survive calcination and the reaction temperatures that the catalyst composite material is exposed to during the method. For example, the binder may be a clay of any type. In particular embodiments that may be mentioned herein, the binder may be a Montmorillonite clay. Examples of Montmorillonite clays that may be mentioned herein include, but are not limited to Montmorillonite K30.

The layered double oxide in the catalyst composite material may take any suitable form. For example, the layered double oxide may be in the form of a plurality of particles (e.g. nanoparticles), that may be distributed homogeneously throughout the catalyst composite material.

The catalyst composite material may be used in any suitable form, depending on the reaction vessel that it is to be situated in. For example, it may be applied as a coating on the walls of a continuous flow device or it may be provided in the form of pellets. In certain examples mentioned herein, the catalyst composite material may be in the form of a pellet.

The catalyst composite material may comprise any suitable amount of the layered double oxide and the binder. For example, the composite catalyst composite material may comprise:
(a) from 30 to 50 wt. % of the layered double oxide; and
(b) from 50 to 70 wt. % of the binder.

While a solvent may be used in the method disclosed herein, it is preferred that no solvent is used. Thus, in embodiments of the invention that may be disclosed herein, step (ii) may be performed in the absence of solvent.

Any suitable feedstock may be used herein. For example, the feedstock may be derived from a fossil fuel and/or be biologically derived (e.g. any associated bio-lipids/fatty acids derived from vegetable (e.g. palm) or animal oils or any other biological source). In certain embodiments that may be mentioned herein, the feedstock may comprise of vegetable fatty acid distillate (VFAD) (e.g. palm fatty acid distillate (PFAD)). In certain embodiments, the feedstock may be PFAD.

When used herein, the term “fatty acid” may refer to any fatty acid material. For example, each fatty acid mentioned herein may be a monocarboxylic acid having from 12 to 22 carbon atoms or a dicarboxylic acid having from 2 to 11 carbon atoms. The fatty acids may be saturated or unsaturated. Additionally or alternatively, the fatty acids may be branched or linear.

The wax obtained from the process may be any suitable wax. For example, the wax may be a bio-wax, a non-bio-wax or a partial bio-wax, depending on the feedstock(s) used. The wax may be a wax having one or more components having from 23 to 43 carbon atoms. For example, the wax may be a bio-wax. In particular embodiments that may be mentioned herein, the wax obtained by the process may be a bio-wax having one or more components having from 23 to 43 carbon atoms.

In particular embodiments that may be mentioned herein, when the wax is formed from a feedstock that only includes monocarboxylic acids, then the wax may have one or more components having from 23 to 43 carbon atoms.

In certain embodiments that may be mentioned herein, step (ii) of the method may be: performed in the absence of a solvent; and/or the binder may be a clay of any type, for example, the clay may be a Montmorillonite clay; and/or the layered double oxides are distributed throughout the catalyst composite material.

The method disclosed herein may be conducted at any suitable temperature. For example, step (ii) of the method may be performed at a temperature of from 350 to 450° C., such as from 380 to 420° C., e.g. about 400° C.

The method disclosed herein may be conducted at any suitable Liquid Hourly Space Velocity (LHSV). For example, step (ii) of the method may be performed at an LHSV of from 0.8 to 1.2.

As will be appreciated, the method disclosed herein operates through the use of a catalyst composite material. Thus in a second aspect of the invention, there is provided a catalyst composite material comprising:
(a) a layered double oxide; and
(b) a binder.

As will be appreciated, embodiments of the catalyst composite material have been described hereinbefore and will not be repeated for the sake of brevity.

In a third aspect of the invention, there is provided a method of forming a catalyst composite material as described herein, wherein the method comprises the steps of:
(a) providing a mixture comprising a layered double oxide, a binder, a plasticizer, a lubricant, and a peptizer; and
(b) subjecting the mixture to calcination for a period of time to provide the catalyst composite material.

The layered double hydroxide (LDH) may be any suitable LDH that can make the layered double oxides mentioned hereinbefore.

The plasticizer may be any suitable plasticizer material. For example, the plasticizer may be a material containing polar and/or non-polar functionality that can be used as a plasticizer. Examples of suitable materials include, but are not limited to water, polyethylene glycol and hydroxyethyl cellulose.

The lubricant may be any suitable lubricant material. For example, the lubricant may be a diol that can be used as a lubricant. Examples of suitable lubricants include, but are not limited to triethyleneglycol, ethylene glycol, glycerin, graphite, mineral oil, propylene glycol and aluminium stearate.

The peptizer may be any suitable peptizer material. For example, the peptizer may be an acid that can be used as a peptizer. Examples of suitable acids may be an inorganic or organic acid, such as an inorganic acid. An inorganic acid that may be disclosed herein include, but are not limited to, nitric acid, hydrochloric acid, sulphuric acid, boric acid and phosphoric acid. An organic acid that may be disclosed herein include, but are not limited to, acetic acid, citric acid and formic acid.

The mixture may comprise:
(a) from 30 to 50 wt. % of the layered double oxide; and
(b) from 50 to 70 wt. % of the binder.

In addition, the mixture may include:
(c) from 5 to 50 wt. % of the plasticizer; and
(d) from 5 to 20 wt. % of the lubricant; and
(e) from 5 to 20 wt. % of the peptizer.

The amounts of materials (c), (d), and (e) are each relative to the 100 wt % of the combination of the layered double oxide and the binder.

Further aspects and embodiments will now be described by reference to the following non-limiting embodiments.

### EXAMPLES

### Materials

Magnesium nitrate hexahydrate and Aluminium nitrate nonahydrate were purchased from Systerm. Urea for synthesis and Montmorillonite K30 were purchased from Sigma-Aldrich. Nitric acid 65% was purchased from EMSURE-Merck. Triethylene glycol 99% was purchased from ACRO Organics. Palmitic acid was purchased from R&M.

### Example 1. Synthesis of Mg—Al Derived Layered Double Oxide (LDO)

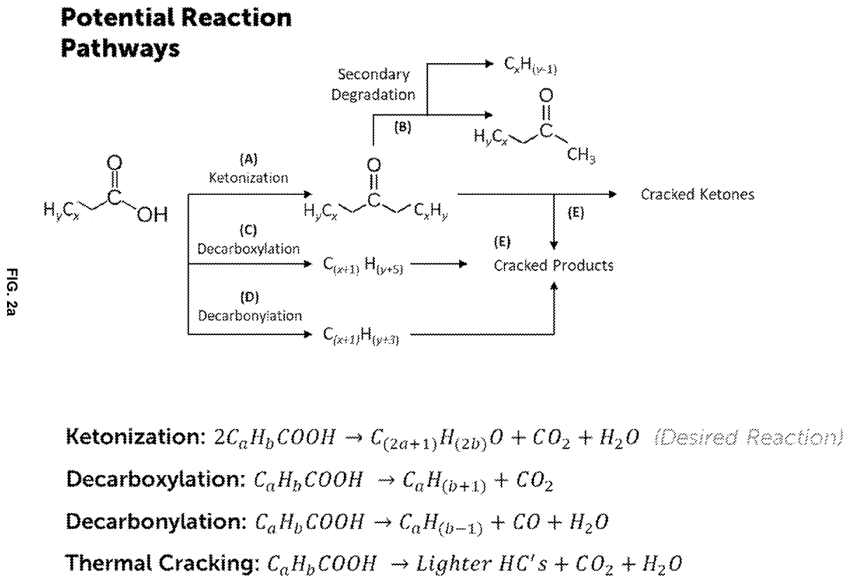

LDO is a representative host material for the synthesis of inorganic nanosheets and commonly derived from the Brucite structure (FIG. 2). The novelty of LDO is the structure of the compound which is a 2D sheet-like morphology that:
increases surface area for high exposure of active site; and
improves the dispersion/distribution of the catalytic active sites within the LDO.

The Mg—Al derived LDO in powder form can be prepared by following any of the procedures below. As will be appreciated, the specific amounts may be derived from the journal articles (and specifically their preparations of LDOs) mentioned below, where relevant, which are hereby incorporated by reference.

### General Procedure for the Synthesis of Mg—Al Derived LDO

A certain amount of magnesium nitrate hexahydrate was dissolved in distilled water. Then, a certain amount of aluminum nitrate nonahydrate was added into the solution under vigorous stirring. Urea was dissolved in distilled water and then added into the first mixture in dropwise method under vigorous stirring. The resulting mixture was transferred into autoclave reactor, heated at temperature ranging from 60° C. to 150° C. for 1 hour-6 hours under stirring. The Mg—Al derived LDH was washed with distilled water and ethanol several times and dried at 100° C. overnight. The resulting materials was calcined in air to form Mg—Al derived layered doubled oxide.

Zhu, B. et al., Water Sci. Technol. 2018, 78, 1179-1188

Magnesium nitrate hexahydrate (10.256 g, 40 mmol) was dissolved in distilled water (60 mL). Aluminum nitrate nonahydrate (3.752 g, 10 mmol) was added into the solution under vigorous stirring. Urea (6.72 g, 112 mmol) was dissolved in distilled water (60 mL) and then added into the first mixture in dropwise method under vigorous stirring. The resulting mixture was transferred into autoclave reactor, heated at 150° C. for 6 hours under stirring. The Mg—Al derived LDH was washed with ethanol several times and dried at 100° C. overnight. Then, the catalyst was calcined at 500° C. for 5 hours in air to form Mg—Al derived layered doubled oxide.

Jiang, B. et al., Catal. Sci. Technol. 2019, 9, 6335-6344

Appropriate amounts of magnesium nitrate hexahydrate and aluminum nitrate nonahydrate were dissolved in distilled water. An alkaline solution consisting of both sodium hydroxide and sodium carbonate was prepared. Both solutions were introduced simultaneously into the recipient with 200 ml water at 25° C. under vigorous stirring. After precipitation, this suspension was kept at room temperature for 15 hours at 80° C. under vigorous stirring. Then, the precipitate was washed repeatedly with deionized water and dried at 100° C. overnight. Then, the catalyst was calcined in air to form Mg—Al derived layered doubled oxide.

Kocik, J. et al., Mol. Catal. 2021, 516, 111946-111956

The catalyst precursor consisting of a metal nitrates solution (Mg/Al) at a certain molar ratio of Mg:Al (e.g. 1:1, 2:1, 3:1 and 4:1) was continuously added into a reactor together with a basic solution (K₂CO₃/KOH). The reaction mixture was kept at synthesis conditions for 1 hour, 60° C., pH 9.5 and 250 rpm. Then, the resulting product was filtered by the press-filtration method, using filter-press with S15N filtration paper plate. Finally, the product paste was washed with deionised water and dried at 65° C. for 12 hours. Then, the catalyst was calcined in air to form Mg—Al derived layered doubled oxide.

### Example 2. Synthesis of Composite Catalyst Containing Mg—Al Derived LDO and Clay

Mg—Al LDO powder (prepared in Example 1) and clay powder (binder) were mixed thoroughly to homogeneity in a desired ratio (e.g. 3:7 or 5:5). An optimal amount of distilled water (plasticizer) and 65% nitric acid (peptizer, inorganic acid) were mixed in a measuring cylinder to prepare an aqueous solution of HNO₃. Then, triethyleneglycol (lubricant, diol) was added and mixed thoroughly. The resulting liquid mixture was added to the powder mixture and mixed thoroughly until a paste was formed. The paste was loaded in an extruder and extruded using a 1 mm diameter die at a speed of 50 rpm. The extrudates were dried at above 100° C. (e.g. 106° C.) overnight and then calcined at 500° C. for 4 hours in air.

The pelletized composite catalyst contains Mg—Al derived LDO as the active component and clay as the binder, mixed with lubricant, plasticizer and peptizer (Table 1).

TABLE 1
Components of the catalysts.

| Components of the catalysts | in wt % |
|---|---|
| Mg—Al derived LDO | 30-50 |
| Clay (binder) | 50-70 |
| Water (plasticizer) | 5-50 |
| Diol (lubricant) | 5-20 |
| Inorganic acid (peptizer) | 5-20 |

The resulting high-performing catalyst in pellet form includes Mg—Al LDO as the active component and a clay-based binder.

In an alternative method, LDH may be used in place of LDO in the above preparation. Upon calcination, the LDH forms LDO.

### Example 3. Analysis and Characterisation of the Catalyst

Mg—Al LDO powder (prepared in Example 1) and the Mg—Al LDO-clay composite catalyst pellet (prepared in Example 2) were characterized.

### FESEM-EDX

Morphological composition of the catalysts was captured via field emission scanning electron microscopy-energy dispersive-ray (FESEM-EDX). The FESEM images were recorded using LEO 1455 VP electron microscope. The sample catalyst was dispersed on an aluminium sample holder using tape. Next, the dispersed sample was coated with a thin layer of platinum as it allows the sample to be imaged without distortion due to charging effect. The EDX was performed by Rayny EDX-720 spectrometer for the determination of elemental composition of the synthesized catalyst.

### Brunauer-Emmett-Teller (BET)

The surface area and pore properties were calculated by the BET using Micromeritics ASAP 2020 model. The catalysts were degassed overnight at 150° C. for 8 h to remove moisture and foreign gases adsorbed on the surfaces of the catalyst. Adsorption and desorption process of N₂ on the catalyst surfaces were analyzed in a vacuum chamber from 50° C. to 900° C. The pore size distribution of catalyst was calculated using the Barrett-Joyner-Halenda (BJH) method.

### Gas Chromatography-Flame Ionization Detection (GC-FID)

A certain amount of sample was added into a 10 mL volumetric flask. 100 μL of internal standard (optionally heptane) was added into the flask, followed by chloroform until the 10 mL mark was reached. The solution was then filtered and transferred into a small GC vial for analysis.

### TPD

Temperature programmed desorption was employed to analyse the acidity and basicity of the synthesized catalysts. The temperature programmed desorption of carbon dioxide (TPD-CO₂) was used to characterize the basic sites of the catalysts using Thermo Finnigan TPD/R/O 1100 (fully equipped with TCD). The catalyst (~0.05 g) was pre-treated by N₂ gas flow for 30 min and at 250° C. Then, the catalyst was exposed to CO₂ gas for 1 hour at ambient temperature to allow adsorption of CO₂ molecules onto the catalyst surfaces. The excess CO₂ is subsequently flushed out with N₂ gas flow at rate 20 mL/min for 30 min. The desorption of CO₂ from the basic sites of the catalyst was detected by TCD under helium gas flow (30 mL/min) from 50° C. to 900° C. and held for 30 min. Similarly, for temperature programmed desorption of ammonia (TPD-NH₃), ammonia was used as the probe gas and the adsorption and desorption method is the same as TPD-CO₂ steps. The desorption peaks resulting from the analysis were used to determine the basicity/acidity of the catalyst.

### Results and Discussion

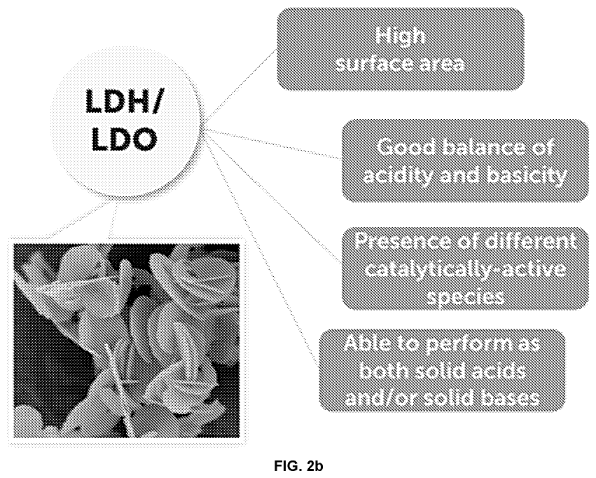

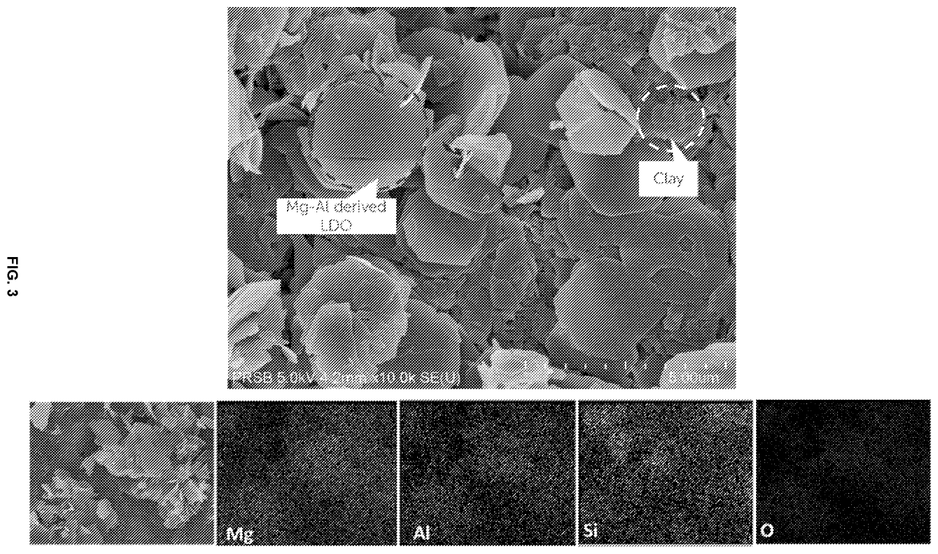

The FE-SEM morphology and EDX mapping of Mg—Al derived LDO-clay composite catalyst pellet are depicted in FIG. 3. FIG. 4 and Table 2 depict the catalyst surface area via BET.

TABLE 2
Catalyst surface area via BET.

| | S_{BET} | Pore Volume | Pore Diameter |
|---|---|---|---|
| Sample | (m² g⁻¹) | (cm³ g⁻¹) | (nm) |
| Mg—Al derived LDO | 22.0 | 0.096 | 17.4 |
| Powder | | | |
| Mg—Al derived LDO-clay | 105.2 | 0.177 | 6.7 |
| composite catalyst | | | |
| pellet (uncalcined) | | | |
| Mg—Al derived LDO-clay | 135.2 | 0.291 | 8.6 |
| composite catalyst | | | |
| pellet (calcined) | | | |

The pelletized catalyst shows larger surface area as compared to its powder form. Calcination helps to increase both the surface area and pore volume of the catalyst.

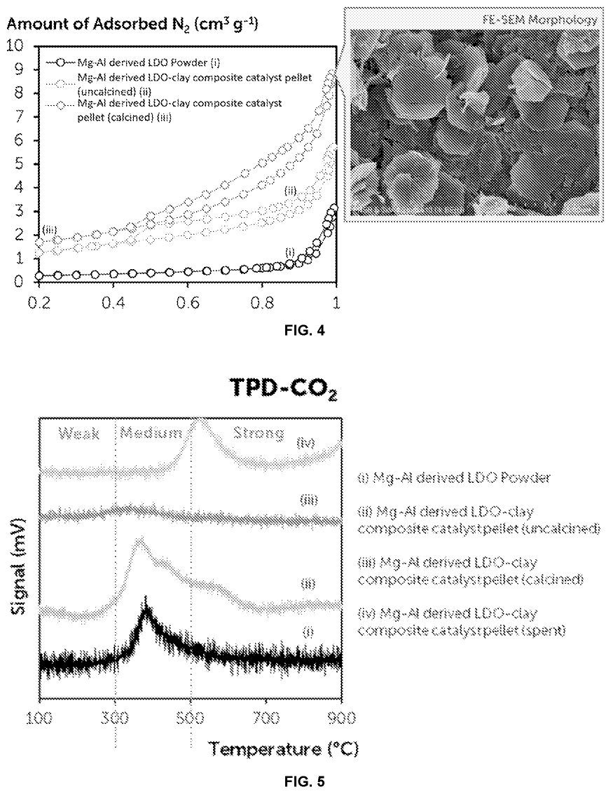

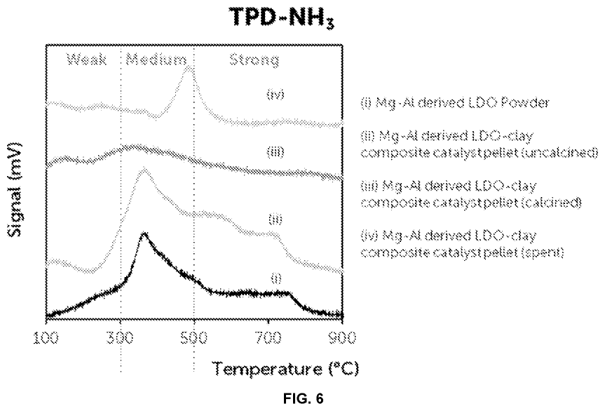

The acidity and basicity for Mg—Al derived LDO-clay composite catalyst via TPD are depicted in FIGS. 5-6. FIGS. 5-6 show that the composite catalyst exhibits moderate strength of acidity/basicity (300-500° C.), which without wishing to be bound by theory may be crucial for feedstock adsorption onto the active sites and desorption off the active sites. The Mg—Al derived LDO-clay composite catalyst shows a distinctive acidic peak at 300-500° C. (intermediate strength acidic sites) as well as a distinctive basicity peak at 300-500° C. (intermediate strength basic sites). Thus, the Mg—Al derived LDO-clay composite catalyst presents a good balance of both acidity and basicity hence the high selectivity towards ketone production (as shown in Example 4).

### Example 4. Catalytic Decarboxylation Reaction of Bio-Derived Fatty Acids

The LDO catalyst has a high ketonization performance. The robustness of the catalyst was tested in the presence of C₁₂-C₂₂ feedstock to yield the desired bio-waxes. As will be appreciated, any suitable fatty acid feedstocks (from any suitable source), so the methodology disclosed herein may be used to make waxes and is not necessarily limited to bio-waxes. In addition to that, dicarboxylic acid having from 2 to 11 carbon atoms can be reacted. Additionally, or alternatively, the fatty acids may be saturated or unsaturated. Additionally, or alternatively, the fatty acids may be branched or linear.

### Catalytic Decarboxylation Reaction of Bio-Derived Fatty Acids in a Continuous Flow Packed Bed Reactor

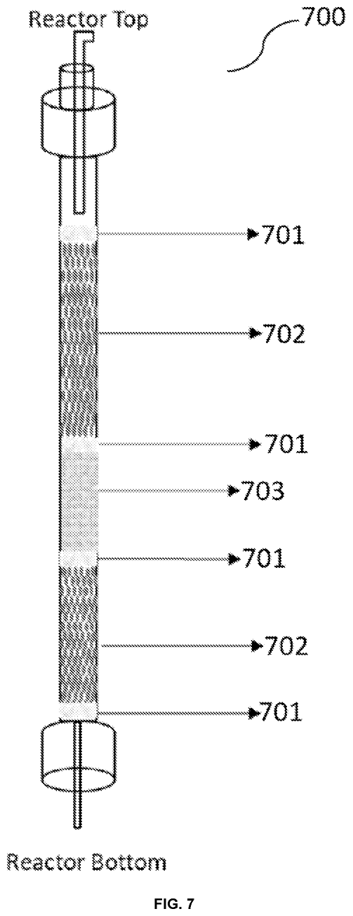

The ketonization of carboxylic acid and any associated bio-lipids/vegetable or animal oil-derived fatty acids was investigated as the main reaction to examine catalyst performance. The reaction was performed in a packed bed continuous flow reactor (FIG. 7). The reactor 700 includes quartz wool 701, support balls 702, and a catalyst and support 703. The reactor is designed for operation up to 550° C. and 100 bar, with heating means of an electric furnace. The reactor was equipped with a pump for feed-in system and a liquid-gas separator at the product section.

A certain amount (e.g. 3 g) of the Mg—Al derived LDO-clay composite catalyst (prepared in Example 2) was loaded into a reactor tube (10 mm internal diameter) together with inert support balls or quartz beads to support the catalyst. The carboxylic acid and any associated bio-lipids/vegetable or animal oils derived fatty acids (e.g. palmitic acid) was melted and fed into the heated feed pump reservoir. The furnace was heated up to a certain reaction temperature (400° C., reaction temperature) gradually and the nitrogen carrier gas was controlled at an optimal flow rate (e.g. a flow rate of 20 mL per hour). The gas outlet valve was opened to vent the gas. Once the desired conditions were reached and stabilized, the feed pump was turned on at an optimal Liquid Hourly Space Velocity (LHSV) (e.g. with a flow rate of 3 mL per hour (LHSV of 1 hour⁻¹)). The liquid product was collected periodically for instance, hourly basis.

In a version of the above-mentioned process, the fatty acid was palmitic acid, resulting in palmitone as the product product.

### Free Fatty Acid (FFA) Conversion Via Titration Method

A portion of the collected liquid product from the above reaction was dissolved in ethanol. Two drops of phenolphthalein were added into the resulting solution. Then, the solution was titrated with 0.01 M potassium hydroxide until the color of the solution turned light pink. The conversion of the FFA remaining was then calculated based on the amount of potassium hydroxide required to achieve the light pink colour.

The reaction was stopped when the reaction had reached completion.

### Results and Discussion

The process involves a solvent-free decarboxylative coupling reaction in which two (or more) carboxylic acid molecules react to form a ketone, together with carbon dioxide and water co-products, as opposed to conventional methods where solvent is required. The feedstock is undiluted prior to feeding into reactor.

As noted above, an example of the process was conducted using palmitic acid, resulting in the generation of palmitone. Table 3 shows the palmitone yield from this decarboxylative coupling reaction.

TABLE 3
Palmitone yield from decarboxylative coupling reactions.

| Catalyst | Palmitone Yield (%) |
|---|---|
| NiI | <6% |
| Clay pellet | <20% |
| Mg—Al derived LDO-clay composite catalyst pellet | >90% |

In addition, the Mg—Al derived LDO-clay composite catalyst pellet was used with a range of other fatty acid feedstocks (C₁₂-C₂₂; optionally of biological origin) to provide waxes (e.g. bio-waxes) having carbon atom chain lengths in the range of C₂₃-C₄₃. In addition to that, dicarboxylic acid having from 2 to 11 carbon atoms can be reacted. Additionally, or alternatively, the fatty acids may be saturated or unsaturated. Additionally, or alternatively, the fatty acids may be branched or linear.

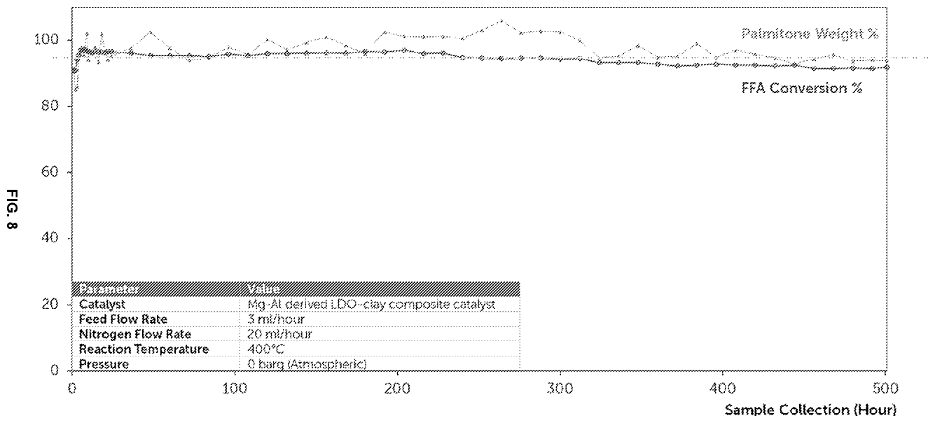

It is believed that the composite material formed from LDO and clay results in unique physicochemical properties to provide high conversion and product selectivity (see FIG. 8 and Table 4).

TABLE 4
Reaction product component distribution.

| Product Component | Weight % |
|---|---|
| Palmitone | >90% |
| Other Ketones | <5% |
| Alkanes & Alkenes | <5% |

Finally, the catalytic performance of Mg—Al derived LDO-clay composite catalyst was rigorously tested in a continuous flow packed bed reactor for >500 hours (FIG. 8), as described in Example 4.

The composite catalyst of LDO and clay provides a material with desirable physicochemical properties, robustness, high catalytic activity and stability. The catalyst exhibits moderate strength of acidity/basicity (in the operating temperature range of 300-500° C.), which may enable the feedstock to be adsorbed and desorbed onto and from the active sites. The presence of CO₂ may deactivate the catalyst by binding on the strong basic sites whereas feedstock adsorption is hindered at the weak basic sites, as such a composite catalyst that avoids such strong and weak sites is more able to provide the desired catalytic activity without deactivation or slow reactivity. Furthermore, the composite catalysts disclosed herein have a high catalytic activity with conversion of ~99% and selectivity towards bio-wax of >90% at reaction temperature of 400° C. and LHSV of 0.8-1.2. In addition, the catalyst performance was sustained for >500 hours without any deactivation.

1. A method of preparing a wax, comprising the steps:
(i) providing a feedstock comprising one or more fatty acids, each of the one or more fatty acids having from 12 to 22 carbon atoms; and
(ii) subjecting the feedstock to a ketonisation reaction to produce a wax, wherein said ketonisation reaction is catalysed by a catalyst composite material comprising:
(a) a layered double oxide; and
(b) a binder.

2. The method according to claim 1, wherein step (ii) is performed in the absence of solvent.

3. The method according to claim 1, wherein the one or more fatty acids are selected from monocarboxylic acids and dicarboxylic acids.

4. The method according to claim 3, wherein when the one or more fatty acids are only monocarboxylic acids, then the wax has one or more components having from 23 to 43 carbon atoms.

5. The method according to claim 1, wherein:
the layered double oxide is in the form of a plurality of particles that are distributed homogeneously throughout the catalyst composite material; and/or
the layered double oxide is in the form of a plurality of particles that are distributed homogeneously throughout the catalyst composite material.

6. The method according to claim 1, wherein the catalyst composite material is in the form of a pellet.

7. The method according to claim 1, wherein the layered double oxide is formed from the combination of divalent metal cations, trivalent metal cations and oxygen atoms, wherein:
the divalent metal cations are selected from one or more of the group consisting of magnesium, copper, zinc, nickel, and manganese; and
the trivalent metal cations are selected from one or more of the group consisting of aluminium, iron, and chromium.

8. The method according to claim 1, wherein the catalyst composite material comprises:
(a) from 30 to 50 wt. % of the layered double oxide; and
(b) from 50 to 70 wt. % of the binder.

9. The method according to claim 1, wherein:
step (ii) is performed in the absence of a solvent; and/or
the binder is a clay of any type.

10. The method according to claim 1, wherein step (ii) is performed at a temperature of from 350 to 450° C.

11. The method according to claim 1, wherein step (ii) is performed at Liquid Hourly Space Velocity (LHSV) of from 0.8 to 1.2.

12. The method according to claim 1, wherein the feedstock is one or more of a fossil fuel-derived feedstock and a biologically-derived feedstock.

13. A catalyst composite material comprising:
(a) a layered double oxide; and
(b) a binder.

14. The catalyst composite material according to claim 13, wherein the binder is a clay of any type.

15. The catalyst composite material according to claim 13, wherein the layered double oxide is in the form of a plurality of particles that are distributed homogeneously throughout the catalyst composite material.

16. The catalyst composite material according to claim 13, wherein the layered double oxide is a magnesium-aluminium layered double oxide.

17. The catalyst composite material according to claim 13, comprising:
(a) from 30 to 50 wt. % of the composite material; and
(b) from 50 to 70 wt. % of the binder.

18. The catalyst composite material according to claim 13, wherein the catalyst composition is in the form of a pellet.

19. A method of forming a catalyst composite material according to claim 13, wherein the method comprises the steps of:
(a) providing a mixture comprising a layered double oxide, a binder, a plasticizer, a lubricant, and a peptizer; and
(b) subjecting the mixture to calcination for a period of time to provide the catalyst composite material.

20. The method according to claim 19, wherein the plasticizer is a material containing polar and/or non-polar functionality.

21-22. (canceled)
